(19) United States
(12) Patent Application Publication
(10) Pub. No.: US 2022/0039708 A1
(43) Pub. Date: Feb. 10, 2022

(54) PULSE OXIMETRY USING AMBIENT LIGHT

(71) Applicant: The Regents of the University of California, Oakland, CA (US)

(72) Inventors: Ana Claudia Arias, Lafayette, CA (US); Donggeon Han, Berkeley, CA (US); Yasser T. Khan, Berkeley, CA (US); Jonathan KangYu Ting, Berkeley, CA (US); Igor Igal Deckman, Albany, CA (US)

(21) Appl. No.: 17/373,345

(22) Filed: Jul. 12, 2021

Related U.S. Application Data
(63) Continuation of application No. PCT/US2020/013496, filed on Jan. 14, 2020.
(60) Provisional application No. 62/792,112, filed on Jan. 14, 2019.

(51) Int. Cl. A61B 5/1455; A61B 5/024; H01L 27/30

(52) U.S. Cl. CPC A61B 5/14552; H01L 51/0097; H01L 27/305; A61B 5/02427

(57) ABSTRACT

Systems and methods to measure pulse and blood oxygen saturation in tissue using pulse oximetry with an ambient light source. Certain pulse oximeters according to various embodiments advantageously do not require and do not include a light source such as an LED, thereby reducing complexity and reducing power consumption.

### CROSS-REFERENCE TO RELATED APPLICATIONS

This patent application claims priority to International Patent Application No. PCT/US2020/013496, entitled, “PULSE OXIMETRY USING AMBIENT LIGHT,” filed Jan. 14, 2020, and to U.S. Provisional Patent Application No. 62/792,112, entitled “PULSE OXIMETRY USING AMBIENT LIGHT,” filed Jan. 14, 2019, which are both incorporated herein by reference.

### STATEMENT REGARDING FEDERALLY SPONSORED RESEARCH OR DEVELOPMENT

This invention was made with Government support under Grant Numbers EEC-1160494 and EECS-1202189 awarded by the National Science Foundation. The Government has certain rights in this invention.

### BACKGROUND

The present disclosure provides systems and methods to measure pulse and blood oxygen saturation in living tissue using pulse oximetry with an ambient light source.

Photoplethysmography (PPG), which is a non-invasive optical technique of detecting blood volume changes in tissue, uses a light source with a light spectrum that can penetrate the tissue, and a light detector that can sense that light. Signal obtained from PPG can provide vital information about the subject including physiological signs (e.g. heart rate, respiration, blood pressure, etc.), vascular condition and heart or cardiovascular variability.

When PPG signals can be obtained from two specific portions of the light spectrum, oxygen saturation, SpO₂, can be estimated by taking a ratiometric measurement of the two signals. This is possible by taking advantage of the different light absorption characteristics of oxyhemoglobin (HbO₂) and deoxyhemoglobin (Hb) at the two light spectrum. Pulse oximetry, a method to obtain SpO₂ is a safe and inexpensive way of measuring oxygen saturation as well as other vital signs mentioned above, and is widely used in clinical use.

Pulse oximeters traditionally consist of light-emitting diodes (LEDs) and photodiodes (PDs) which can operate at two different wavelength spectrum. The two spectrum can either be green and red, or red and near-infrared (NIR), where the acceptable spectrum range is given (in nanometers) by 470<green<550, 620<red<690, and 740<NIR<950. Preferably, overlap between the two spectrum should be minimized for better accuracy in oxygen saturation calculation. Usually, a photodiode that can sense broad range of spectrum is selected and combined with two LEDs which can provide two different localized light spectrum. Most oximeters have been designed in this two LEDs, one PD (2L1P) setup. With this scheme, the photodiode itself cannot distinguish the wavelength of the incoming light. The two LEDs must operate in turn at a given frequency where the photodiode is synchronized accordingly, and the wavelength of the light detected corresponds to that of the synchronized LED. This method needs an ambient light calibration scheme in order to reduce the effect of ambient light to the signal. In addition, the operation of LEDs accounts for significant amount of the power consumed by the pulse oximeter and numerous approaches have been suggested to reduce the power consumed by the LEDs, such as reducing the duty ratio or intermittently turning off the LEDs. A one LED and two PDs (1L2P) concept of detecting changes in the tissue oxygenation was also previously demonstrated. This scheme uses a wide spectrum LED which has both red and NIR components and relies on PDs with filters to distinguish the spectrum. Although the two PDs that were used had non-negligible spectrum overlap which limits their usage in cases where precise measurements are required, relative variation in the tissue oxygenation was successfully observed. Also, this concept can bring improvement in the pulse oximeter design, in that there is only one LED to operate. Nonetheless, for both 2L1P and 1L2P schemes, the fact that LEDs are needed, and that they will drain power remains the same. Also, the LEDs need to be controlled by a LED driver which will require additional components in the front end and hence add complexity to the system.

### SUMMARY

Systems and methods for performing pulse oximetry may be performed with no controlled sources such as LEDs needed. The systems and methods herein may utilize ambient light as a light source, and use spectrally-selective sensors such as spectrally-selective organic photodiodes (OPDs). In certain embodiments, flexible and/or stretchable electronics may be used.

Flexible and stretchable electronics are well suited for wearable sensing and medical monitoring applications, in that they form conformal contact with human body. This provides better SNR compared to rigid electronics, and also allows them to be easily integrated into garments or accessories. Pulse oximetry can also benefit from using flexible optoelectronics. When optoelectronics are well-conformed to the skin, quality of the acquired signal can be greatly enhanced. The use of flexible organic PDs (OPDs) for PPG measurements have been shown to reduce noise current from ambient light considerably. OPDs also demonstrate other advantages such as light weight, decreased fabrication complexity, and mechanical flexibility. These are all useful characteristics of components for wearable and portable applications, which makes OPD an ideal candidate.

One of the distinguished traits that organic absorbers possess is that their spectral sensitivities are relatively narrow, compared to inorganic counterparts. For example, silicon photodiodes are broadband and require carefully designed rigid band-pass filters in order to have good spectral selectivity. Most organic materials are blind in the infrared region and have partial absorption in the visible spectrum. This means that they inherently possess spectral cutoff within or near the visible spectrum, which can be utilized to realize spectral selectivity.

Prior art pulse oximeters that have been presented have operated using one or two LEDs, depending on which scheme was used; 1L2P or 2L1P.

According to various embodiments, pulse oximetry may be performed with no controlled LEDs needed, utilizing ambient light as a light source, and using two spectrally-selective OPDs (e.g., 0L2P) absorbing in red and green or red and NIR wavelengths. Organic absorbers are selected so that the fabricated OPDs will be able to sense green, red, and NIR. In some embodiments, bulk heterojunction blends of poly[N-9″-hepta-decanyl-2,7-carbazole-alt-5,5-(4′,7′-di-2-thienyl-2′,1′,3′-benzothiadiazole)] (PCDTBT) with [6,6]-phenyl C70-butyric acid methyl ester (PCBM70), or poly(3-hexylthiophene) (P3HT) with (O-IDTBR) are used. The OPDs are flexible and compatible with roll-to-roll printing techniques. In some embodiments, these OPDs are combined with appropriate flexible filters that allow the OPDs to become spectrally selective OPDs (ss-OPDs), e.g., to sense green, red or NIR regions with minimum spectral overlap. Using ss-OPDs, it is possible to obtain PPG signals from various ambient lights sources, including sources such as the Sun, or broadband fluorescent, LED and incandescent light sources. For example, in an embodiment, two different ss-OPDs may be used together to perform pulse oximetry under the Sun in the outdoors. In certain embodiments, this system can be integrated into a glove form factor using a wireless data transmission.

According to an embodiment, a heart rate measuring device which can conduct photoplethysmography (PPG) measurements under broadband light is provided that includes a photodiode which has spectrally sensitive sensitivity to a visible wavelength or an infrared wavelength, or both, that penetrate in to skin and reach a pulsating vein.

According to another embodiment, a heart rate measuring device which can conduct photoplethysmography (PPG) measurements under broadband light is provided that consists essentially of: a) a single photodiode that has sensitivity in or to both a first visible light wavelength and a second visible light wavelength, or the first visible light wavelength and an infrared light wavelength, which wavelengths penetrate into the skin and reach a pulsating vein; or b) a first photodiode that has sensitivity in or to the first visible light wavelength, and a second photodiode that has sensitivity in the second visible light wavelength or the infrared wavelength.

According to an embodiment, an oximeter device (e.g., pulse oximeter device) for conducting pulse oximetry measurements using broadband light is provided that includes a first spectrally-selective photodiode (ss-PD) which can sense only red light wavelengths wherein the extinction coefficient ratio of oxy- and deoxy-hemoglobins (ε_{Hb}/ε_{HbO2}) has a value larger than 6; and a second ss-PD which can sense incoming only green light wavelengths, or only NIR wavelengths, wherein the extinction coefficient ratio of oxy- and deoxy-hemoglobins has a value smaller than 2 or 3, respectively.

According to another embodiment, a pulse oximeter device for conducting PPG measurements using broadband light is provided that includes a first spectrally-selective organic photodiode (ss-OPD) comprising a first spectral filter overlaying a sensing region of a first organic photodiode (OPD), wherein the first OPD absorbs/detects light in a first wavelength range including visible wavelengths up to but not including NIR wavelengths, and the first spectral filter only transmits light having wavelengths including and greater than red wavelengths; and a second ss-OPD comprising a second spectral filter overlaying a sensing region of a second OPD, wherein the second OPD absorbs/detects light in a second wavelength range including green wavelengths or NIR wavelengths, and the second spectral filter only transmits light having green wavelengths or light having a wavelength of greater than red wavelengths.

In certain aspects, the pulse oximeter device further includes a flexible substrate, wherein the first ss-OPD and the second ss-OPD are disposed on the flexible substrate. In certain aspects, the first OPD comprises PCDTBT:PCBM70 and the second OPD comprises PCDTBT:PCBM70 or P3HT:O-IDTBR. In certain aspects, the first OPD absorbs/detects light in the first wavelength range including visible wavelengths up to about 700 nm, and wherein the first spectral filter only transmits light having a wavelength greater than about 590 nm. In certain aspects, the second OPD absorbs/detects light in the second wavelength range including visible wavelengths up to about 700 nm, and wherein the second spectral filter only transmits light in a wavelength range of from about 490 nm to about 570 nm. In certain aspects, the second OPD absorbs/detects light in the second wavelength range including wavelengths above about 700 nm up to about 800 nm, and wherein the second spectral filter only transmits light having a wavelength of greater than about 700 nm.

According to yet a further embodiment, a pulse oximeter device for conducting PPG measurements using broadband light is provided that includes a first organic photodiode (OPD) having a first sensing region, the first OPD absorbs/detects light in a first wavelength range including visible wavelengths up to but not including NIR wavelengths; a first optical filter disposed proximal to the first sensing region, the first optical filter transmitting only light having wavelengths including and above red wavelengths; a second OPD having a second sensing region the second OPD absorbs/detects light in a second wavelength range including green wavelengths or NIR wavelengths; and a second optical filter disposed proximal to the second sensing region, the second optical filter transmitting only light including green or only light having a wavelength of greater than red wavelengths. In certain aspects, the first OPD comprises PCDTBT:PCBM70 and the second OPD comprises PCDTBT:PCBM70 or P3HT:O-IDTBR.

According to still a further embodiment, a pulse oximeter device for conducting PPG measurements using broadband light is provided that includes a first organic photodiode (OPD) having a first sensing region, the first OPD absorbs/detects light in a first wavelength range including visible wavelengths up to about 700 nm; a first optical filter disposed proximal to the first sensing region, the first optical filter transmitting light having a wavelength of greater than about 590 nm; a second OPD having a second sensing region, the second OPD absorbs/detects light in a second wavelength range including visible wavelengths and NIR wavelengths greater than 700 nm, e.g., up to about 800 nm or greater; and a second optical filter disposed proximal to the second sensing region, the second optical filter transmitting only light in a wavelength range of from about 490 nm to about 570 nm, or only light having a wavelength of greater than about 700 nm. In certain aspects, the first OPD comprises PCDTBT:PCBM70 and the second OPD comprises PCDTBT:PCBM70 or P3HT:O-IDTBR.

According to another embodiment. a pulse oximeter device for conducting PPG measurements using broadband light is provided that includes a first organic photodiode (OPD) having a first sensing region, the first OPD absorbs/detects light in a first wavelength range including visible wavelengths up to about 700 nm; a first optical filter disposed proximal to the first sensing region, the first optical filter transmitting only light having a wavelength of greater than about 590 nm; a second OPD having a second sensing region, the second OPD absorbs/detects light in a second wavelength range including visible wavelengths; and a second optical filter disposed proximal to the second sensing region, the second optical filter transmitting light only in a wavelength range of from about 490 nm to about 570 nm. In certain aspects, the first OPD comprises PCDTBT:PCBM70 and the second OPD comprises PCDTBT:PCBM70.

According to still a further embodiment, a pulse oximeter device for conducting PPG measurements using broadband light is provided that includes a first organic photodiode (OPD) having a first sensing region, the first OPD absorbs/detects light in a first wavelength range including visible wavelengths up to about 700 nm; a first optical filter disposed proximal to the first sensing region, the first optical filter transmitting only light having a wavelength of greater than about 590 nm; a second OPD having a second sensing region the second OPD absorbs/detects light in a second wavelength range including NIR wavelengths; and a second optical filter disposed proximal to the second sensing region, the second optical filter transmitting only light having a wavelength of greater than about 700 nm. In certain aspects, the first OPD comprises PCDTBT:PCBM70 and the second OPD comprises P3HT:O-IDTBR.

According to another embodiment, method of performing PPG measurements using any pulse oximeter device embodiment herein is provided. The method typically includes positioning the pulse oximeter device on a region of interest of a human user; and exposing the pulse oximeter device to broadband light. In certain aspects, exposing includes irradiating with a broadband light source selected from the group consisting of a fluorescent lamp, an incandescent lamp, and one or more LEDs. In certain aspects, the exposing includes exposing the pulse oximeter device to sunlight.

In certain aspects, the pulse oximeter device elements are arranged on a flexible substrate. In certain aspects, the flexible substrate comprises polyethylene napthalate (PEN) or other flexible polymer or non-polymer material.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1A illustrates a device according to an embodiment receiving abundant amounts of red, green and NIR from sunlight.

FIG. 1B shows the spectrum of the Sun at the global standard spectrum (AM1.5G).

FIG. 1C shows EQE for each of the Green, Red and NIR sensors.

FIG. 1D shows a device structure of an OPD according to an embodiment.

FIG. 1E shows OPDs in FIG. 1D combined with spectral filters.

FIG. 1F shows a schematic top view of a completed sensor system according to an embodiment.

FIG. 2A shows the optical characteristics of the components for Green and Red sensors.

FIG. 2B shows the optical characteristics of the components for the NIR sensor.

FIGS. 3A, 3B and 3C show optical and electrical characteristics of OPDs fabricated with PCDTBT:PCBM70 (S1) or P3HT:O-IDTBR (S2).

FIG. 3D shows the linear dynamic response of the sensors under green, red and NIR LED light.

FIG. 4A shows a sensors on an index finger under a light source.

FIG. 4B shows the spectrum of the Sun at the global standard spectrum (AM1.5G) with the shaded areas provided to aid readers so that the sensitivity regions of the Green, Red and NIR sensors are easily distinguished (and named G, R and N regions for convenience).

FIG. 4C shows the spectrum of fluorescent room light.

FIG. 4D shows the spectrum of LED bulb light (Torchstar A19, 5000K).

FIG. 4E shows the spectrum incandescent light.

FIGS. 5A and 5B show pulse oximetry measurements: the measurements done with the Green and the Red sensor is shown in FIG. 5A, and with the Red and NIR sensors in FIG. 5B; the PPG signals collected from the two sensors are shown in the top two panels.

FIGS. 6A and 6C show readings collected by the prior art oximeter probe.

FIGS. 6B and 6D show readings collected by a present sensor embodiment.

### DETAILED DESCRIPTION

The present disclosure provides systems and methods to measure pulse and blood oxygen saturation in tissue using pulse oximetry with an ambient light source. In certain aspects, the pulse oximeters according to various embodiments advantageously do not require and do not include a light source such as an LED, thereby reducing complexity and reducing power consumption.

### System Design for Pulse Oximetry Using Ambient Light

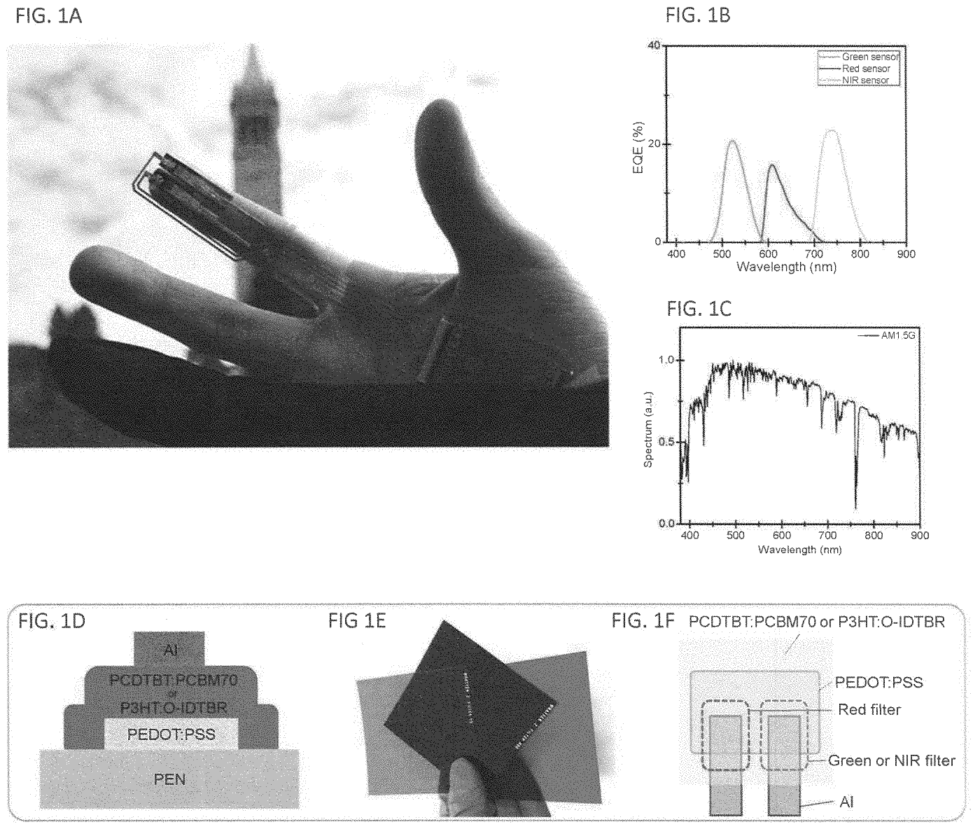

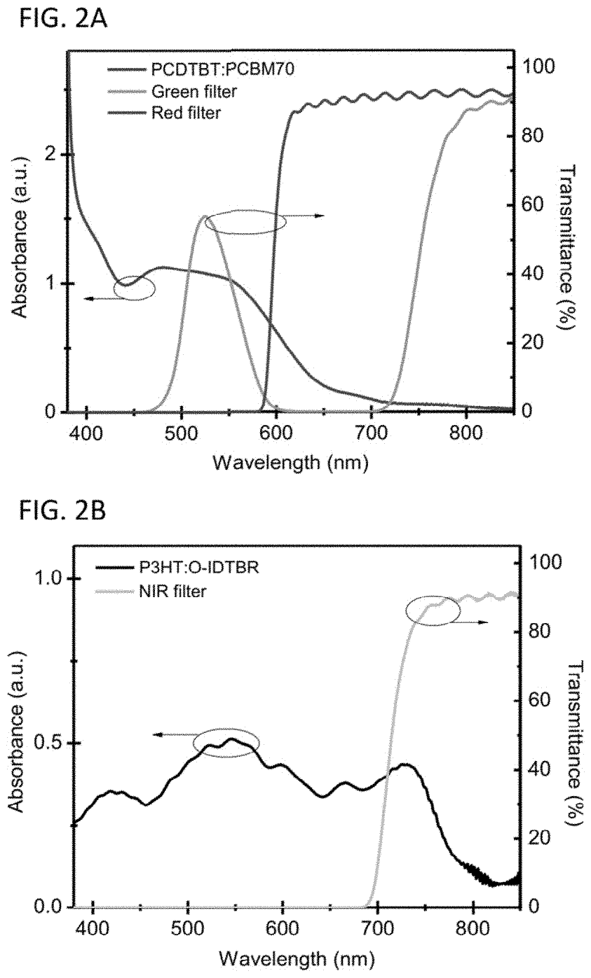

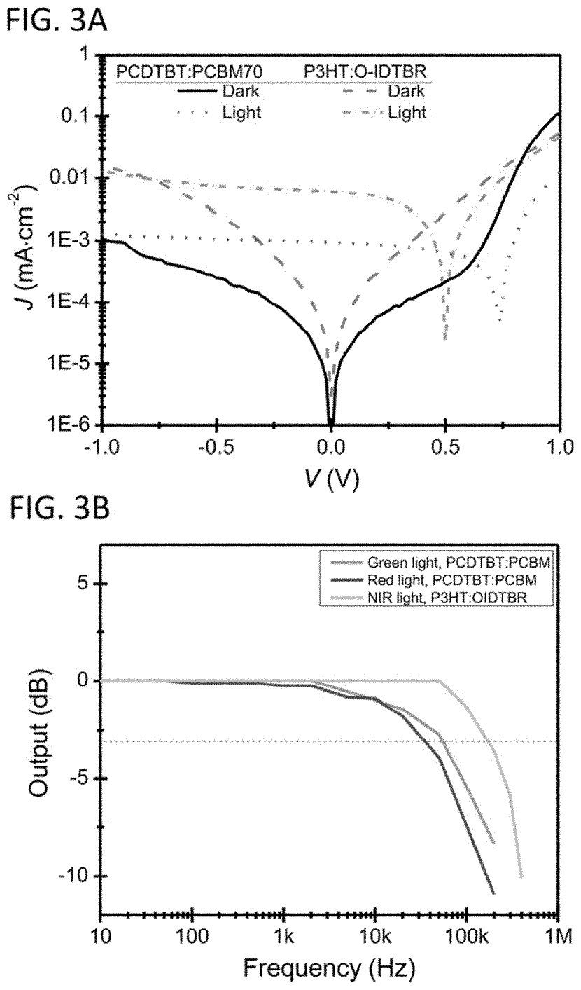

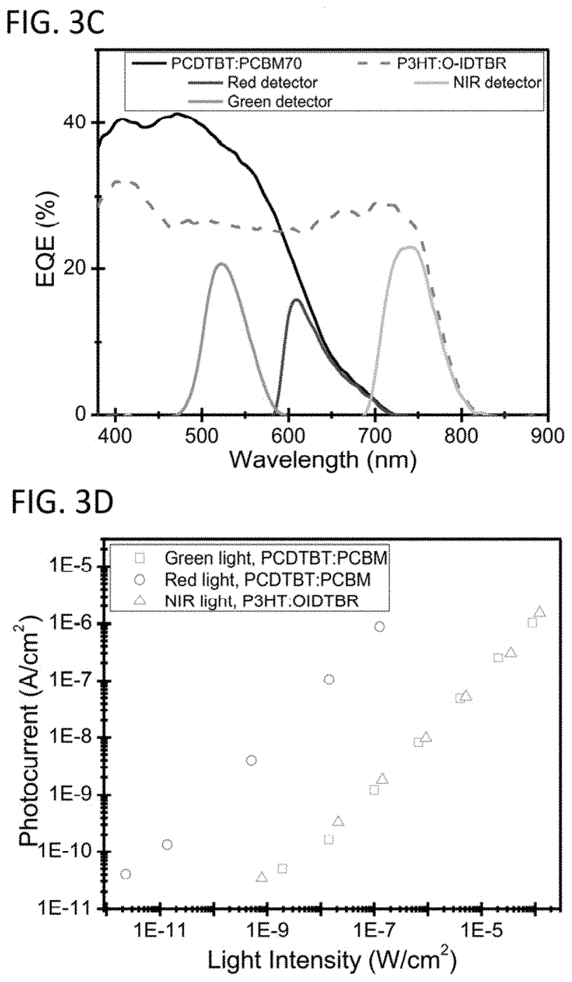

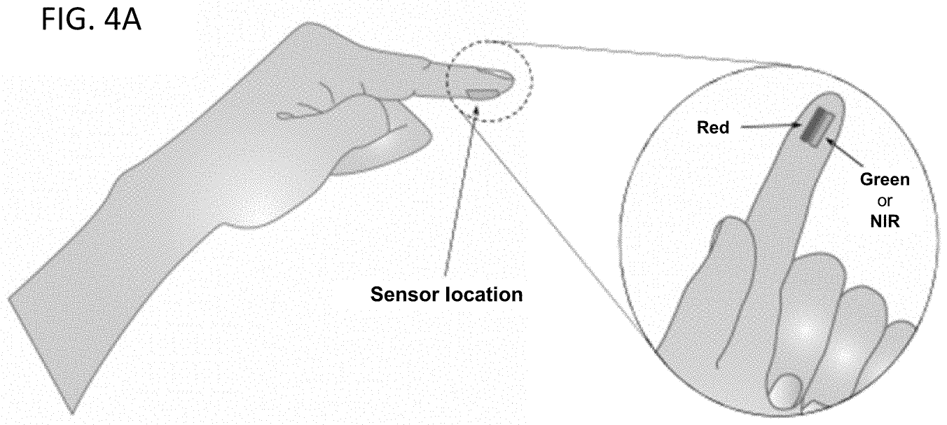

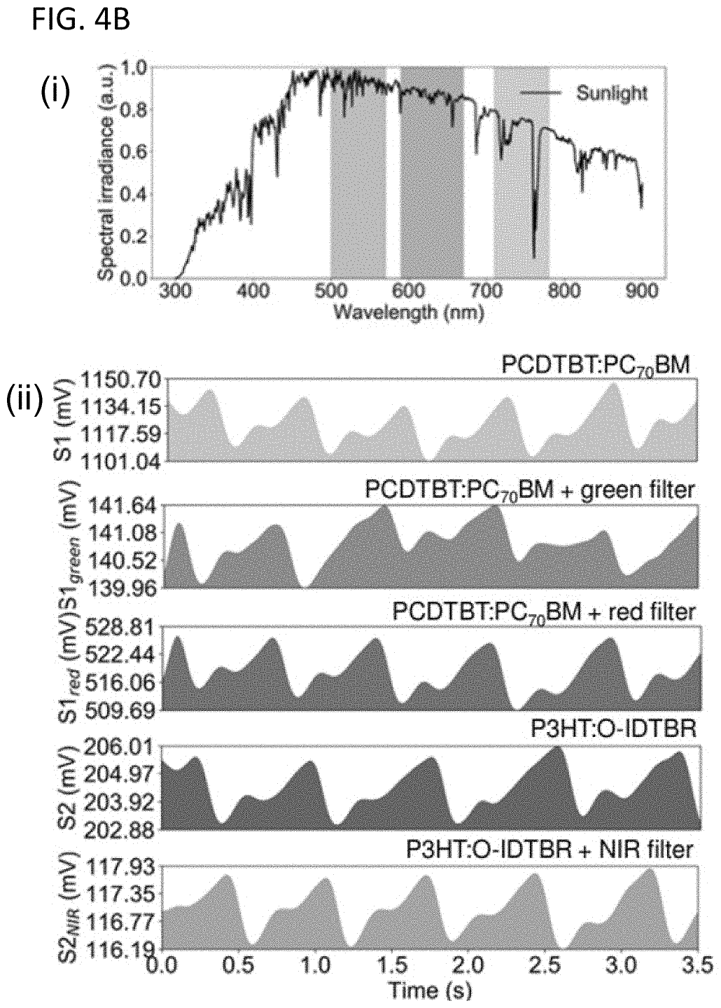

FIG. 1D shows a device structure of an OPD according to an embodiment. Although the spectral sensitivities of organic absorbers are relatively selective, the spectra of OPDs are usually not narrow enough to distinguish the color of the incoming light. In order to make the spectral sensitivities of OPDs narrower, and to make sure there are minimum overlap between different spectral OPDs, the OPDs in FIG. 1D may combined with spectral filters shown in FIG. 1E. This makes the OPDs sense distinctly different spectral regions, which makes them ss-OPDs. External quantum efficiencies (EQEs) spectra of three ss-OPDs are given in FIG. 1C, where green, red and NIR can be sensed, respectively. FIG. 1F shows the schematic top view of the completed sensor system, which includes two ss-OPDs which are placed 2 mm apart where one of the ss-OPDs senses red and the other one senses either green or NIR. When two different PPG signals are collected from each ss-OPDs, pulse oximetry may be performed. For this, any light source containing red and green or MR can be utilized. The Sun is a natural source of light that can provide abundant amounts of red, green and NIR (FIG. 1A). FIG. 1B shows the spectrum of the Sun at the global standard spectrum (AM1.5G). FIG. 1C shows EQE for each of the Green, Red and NIR sensors.

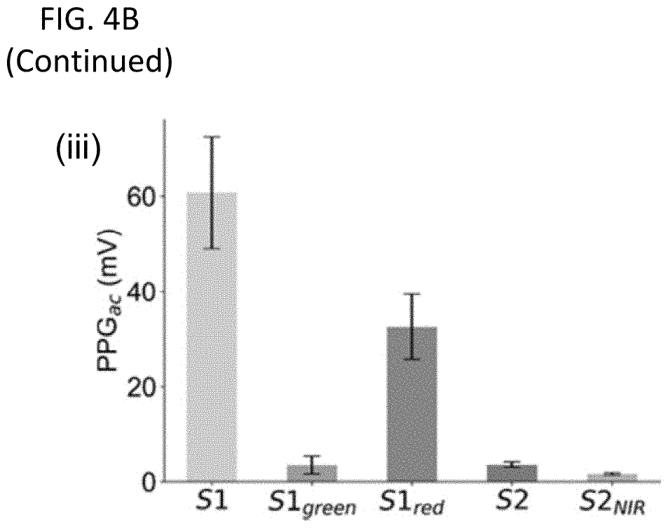

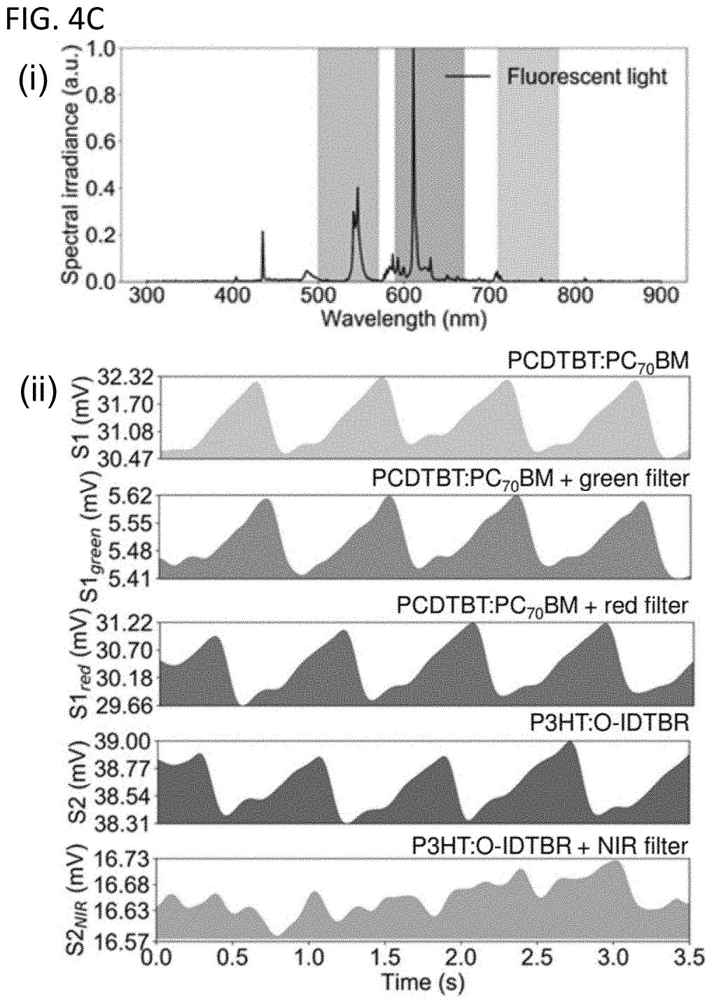

In order to achieve green, red and NIR ss-OPDs (referred to as Green, Red and NIR sensors henceforth), organic photoactive layers and filters are carefully paired by considering their optical characteristics in FIG. 2A and FIG. 2B. In an embodiment, PCDTBT:PCBM70 is chosen as the photoactive layer for Green and Red sensors, since it is known to have absorption in green and red region. It is also widely used for studies in organic solar cells or OPDs due to its stability and reproducibility. FIG. 2A shows the optical characteristics of the components for Green and Red sensors. Blade-coated PCDTBT:PCBM70 film absorbs most of the visible spectrum, showing a decrease in absorbance starting from around 600 nm and up to 700 nm. Absorbance in the NIR is negligible. When this spectrum is overlapped with transmittance of a Green filter (e.g., Kodak 58, Optical Wratten Filter), only the green portion ranging from 490 nm to 570 nm with a peak at 525 nm, which is solely defined by the Green filter, will be absorbed. The NIR portion (>715 nm) of the filter transmittance will be eliminated, since there is no absorbance in PCDTBT:PCBM70. The Red filter (e.g., Kodak 25, Optical Wratten Filter) transmits light starting from 590 nm, all the way up to NIR. When this is combined with PCDTBT:PCBM70, the red portion starting from 590 nm to 700 nm will be sensed, where the lower spectrum region is defined by the filter and the upper region by the photoactive layer. For a NIR sensor however, PCDTBT:PCBM70 cannot be used since it does not absorb in the NIR. In an embodiment, P3HT:O-IDTBR is used as it is capable of absorbing beyond the visible spectrum to the NIR. FIG. 2B shows the optical characteristics of the components for the NIR sensor. Blade-coated P3HT:O-IDTBR film absorbs all of the visible spectrum and partially absorbs NIR, with cut-off wavelength of around 800 nm. The NIR filter (e.g., Kodak 89b, Optical Wratten Filter) is not transmissive in the visible region and starts transmitting from 700 nm. Combining the two will give NIR sensitivity from about 700 nm to about 800 nm.

### Characteristics of Organic Photodiodes

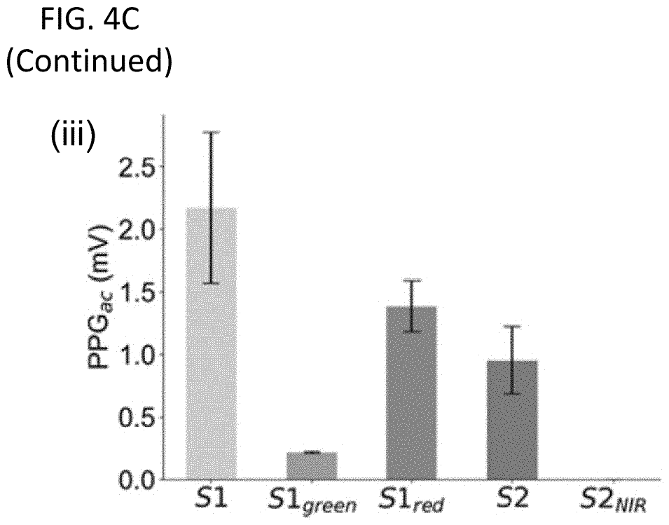

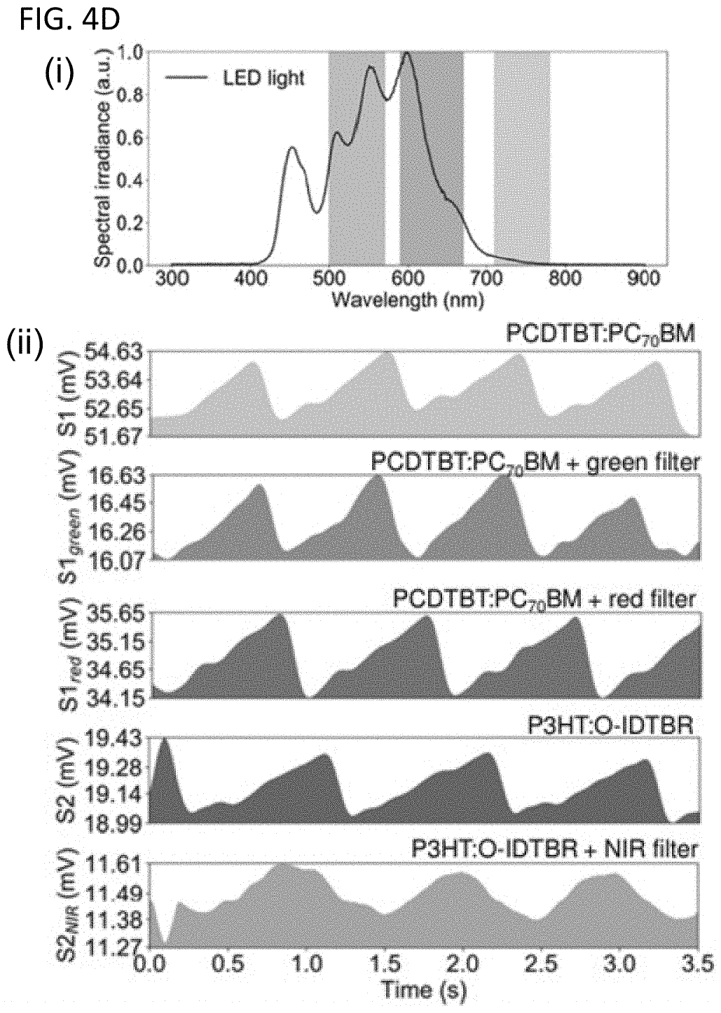

FIGS. 3A-C show optical and electrical characteristics of OPDs fabricated with PCDTBT:PCBM70 (S1) or P3HT:O-IDTBR (S2). The OPDs must meet the following two conditions in order to pick up the pulse: they must be sensitive enough to detect low light intensities which go through the finger, and they must possess adequate frequency response to properly detect timely changes in the blood volume. In FIG. 3A, both OPDs exhibit high reverse bias dark leakage current, which rises with stronger bias. The dark current is minimum at 0V, which is the short circuit condition. Photocurrent under light conditions are also shown. The frequency responses of each OPD are shown in FIG. 3B. The 3 dB frequencies are above 1 kHz for both OPDs, which indicates that the OPDs are suitable for picking up ppg signals. The shape of the EQE spectra in FIG. 3C are similar to the photoactive layer absorption characteristics shown in FIGS. 2A,B; sensitivity of PCDTBT:PCBM70 and P3HT:O-IDTBR based OPDs extending to 700 nm and 800 nm, respectively. The EQE of the Green, Red and NIR sensors are also shown. As described in FIGS. 2A,B, Green and Red sensors are assembled by covering the light incident side of the PCDTBT:PCBM70 based OPDs with either the Green or the Red filters and NIR sensor by covering the P3HT:O-IDTBR based OPD with the NIR filter. The resulting Green, Red and NIR sensors are spectrally selective having sensitivity peaks at 525 nm, 610 nm and 740 nm, respectively, minimal spectral overlap. Overall, through the characterization, it is confirmed that these OPDs are suitable for the purpose of this work. FIG. 3D shows the linear dynamic response of the sensors under green, red and NIR LED light.

### PPG Measurement

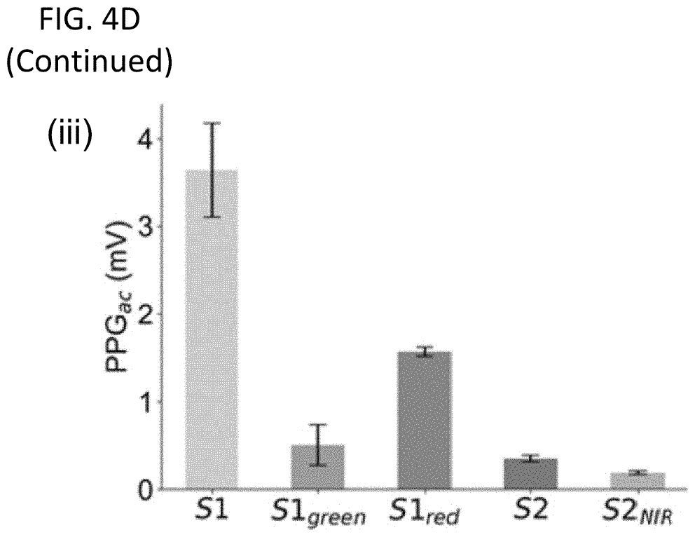

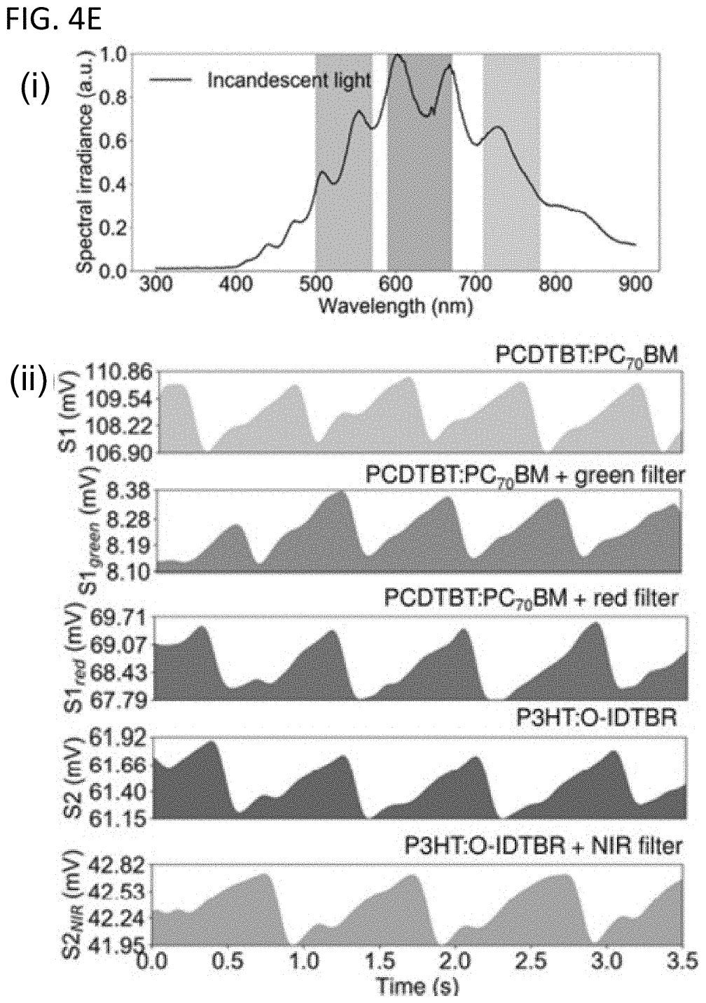

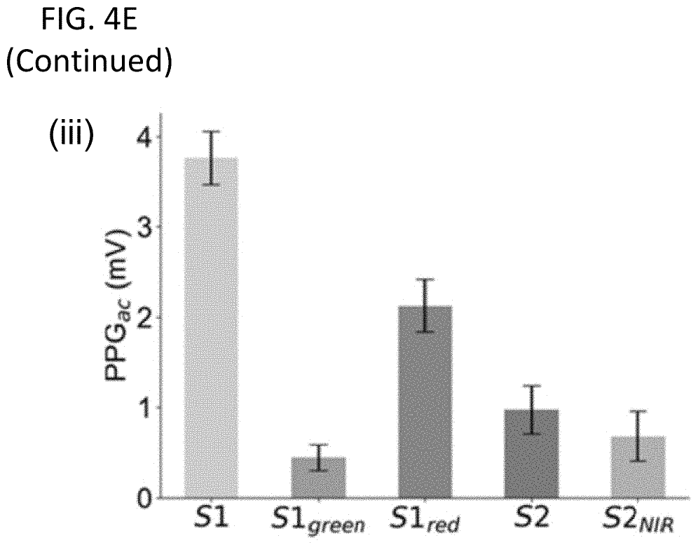

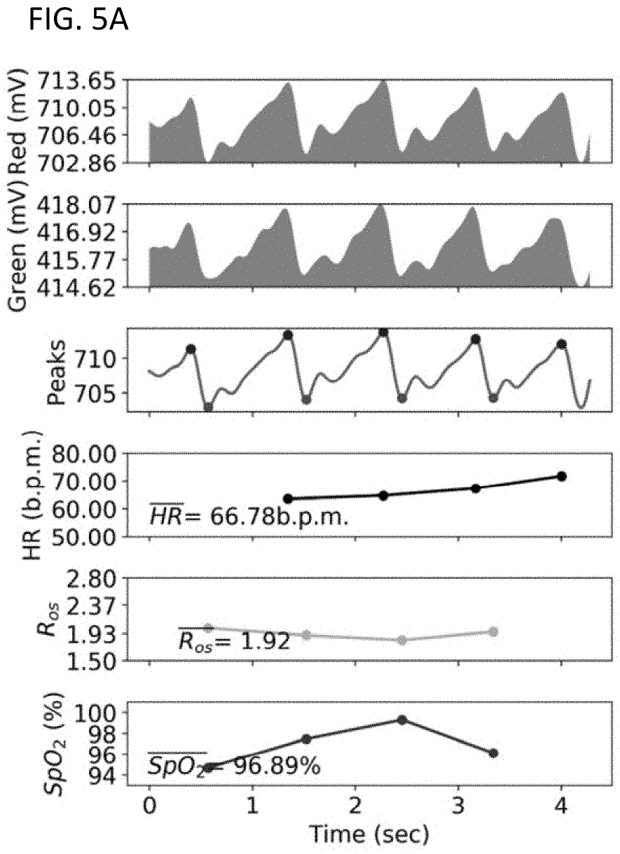

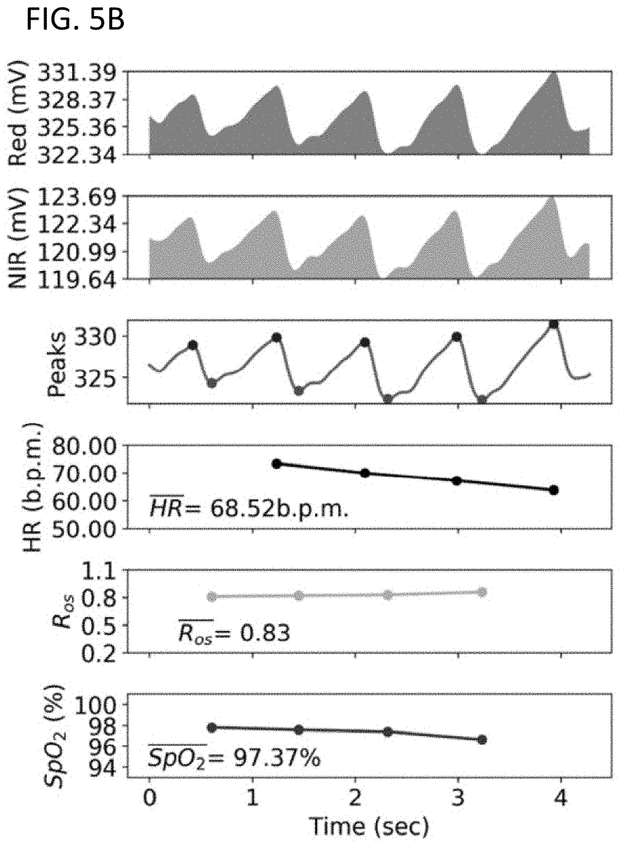

To verify that the OPDs can take PPG measurements using an ambient light source, PPG signals from various ambient light sources were recorded using the sensors. As shown in FIG. 4A a volunteer put on one of the sensors on an index finger under a light source where the readings of the sensor are recorded in a timely manner. The light sources that were tested include the Sun, as well as fluorescent, incandescent and LED lights. FIG. 4B shows the spectrum of the Sun at the global standard spectrum (AM1.5G) with the shaded areas provided to aid readers so that the sensitivity regions of the Green, Red and NIR sensors are easily distinguished (and named G, R and N regions for convenience). The Sun has abundant amount of G, R and N regions altogether. PPG signals taken outdoors under the actual Sun using OPDs based on PCDTBT:PCBM70 without filters (S1), with green filter (Green), with red filter (Red) as well as OPDs based on P3HT:O-IDTBR without filters (S2) and with NIR filter (NIR) are shown sequentially in FIG. 4B. All of the sensors are able to provide PPG signals, which means that both Red+Green and Red+NIR sensor combinations can be used to perform pulse oximetry. Fluorescent light measurement is taken from an office desk where the room is lit by fluorescent lamps, and the distance between the measurement position and the light source is approximately 2 m. The spectrum of fluorescent room light is shown in FIG. 4C. The light has peaky spectrum with two major peaks at 546 nm and 611 nm. 546 nm peak is in the G region and 611 nm in the R region. There is no visible contribution from the N region. This is reflected in the PPG measurements in FIG. 4C. Clear PPG signals can be obtained using 51, Red, Green and S2 sensors, but not with the NIR sensor. A desk lamp is used to provide incandescent and LED light. the distance between the measurement position and the light source is 20 cm. FIG. 4D shows the spectrum of the LED bulb light (Torchstar A19, 5000K). The spectrum includes the G and the R regions with very small contribution in the N, which is again confirmed by the PPG measurements in FIG. 4D. The signals from 51, Green, Red and S2 are clear. There seems to be a small signal picked up from the NIR sensor, but the waveform is not clear. The spectrum of the incandescent light is shown in FIG. 4E. It includes all G, R and N regions and therefore all of the sensors are giving PPG signals in FIG. 4E. As a result, it is confirmed that it is possible to obtain PPG signals from all four light sources, and theoretically all of them can be used to perform pulse oximetry. Irradiance of the indoor light sources used for the measurements are 0.35, 8.3 and 3.7 mW/cm² respectively for fluorescent, incandescent and LED light. The signal magnitudes of the PPG signals obtained from four different light sources using five different OPD+filter combinations are compared in FIGS. 4B-E. Except for the signal obtained from the Sun, the signal magnitudes obtained using S1+filter combinations from the light sources are similar. Even taking into account the variability of each measurement, such as placement location of the sensor or how firmly the sensors were attached to the skin, this is surprising since the difference in irradiance magnitudes is noticeable. Finding the root cause of this will require a systematic study and is out of the scope of this paper. Through this experiment it is confirmed that pulse oximetry can be performed with any of the four ambient light sources, although magnitudes of signals may vary.

### Pulse Oximetry Under the Sun

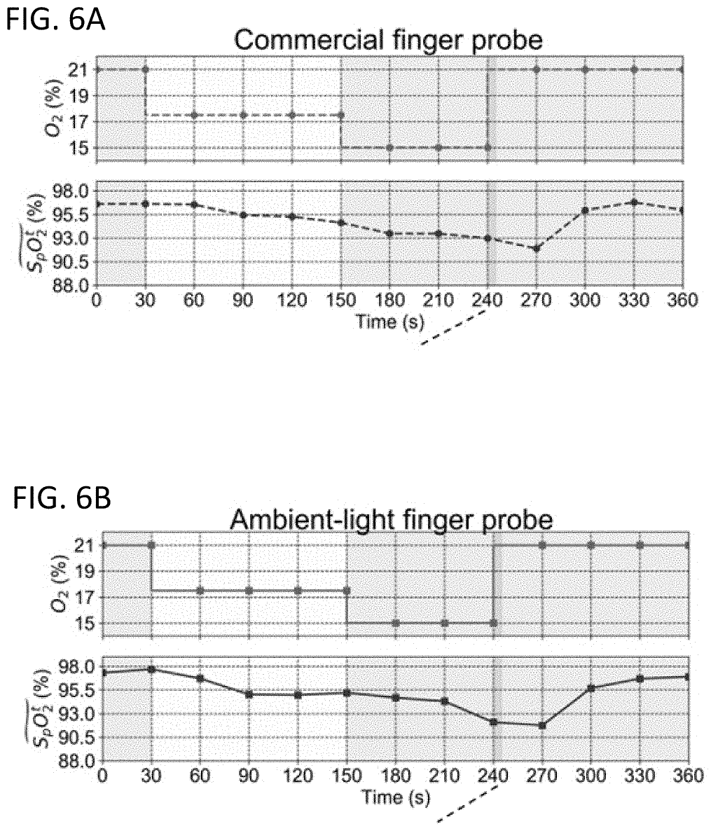

Pulse oximetry was performed under the actual Sun in the outdoors. As was previously mentioned, pulse oximetry can be done either in green and red or red and NIR spectrum. One of the two combinations are placed on a volunteer's index finger and the readings of each sensor are recorded. FIGS. 5A,B shows the pulse oximetry measurement. The measurement done with the Green and the Red sensor is shown in FIG. 5A, and with the Red and NIR sensors in FIG. 5B. The PPG signals collected from the two sensors are shown in the top two panels. Heartbeat peaks and valleys are detected from the PPG signals and the heart rate is calculated.

Pulse Oximetry with Varying Oxygen Saturation

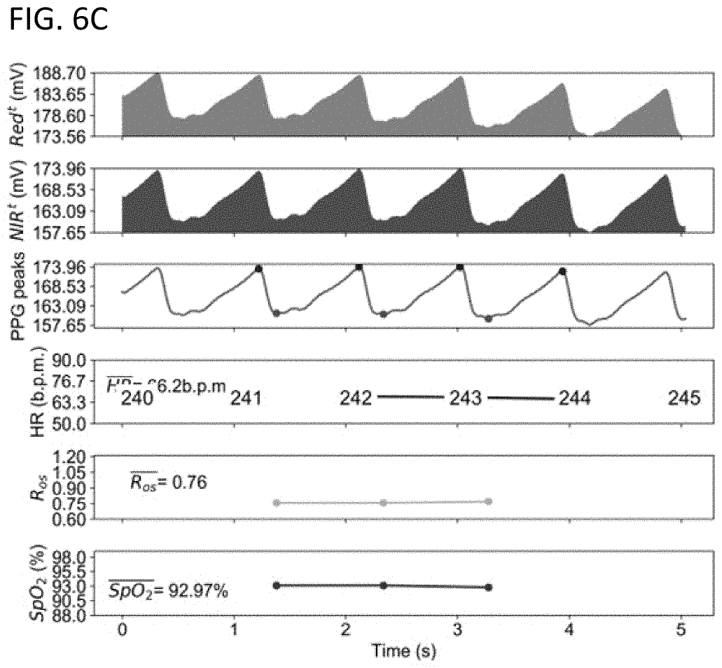

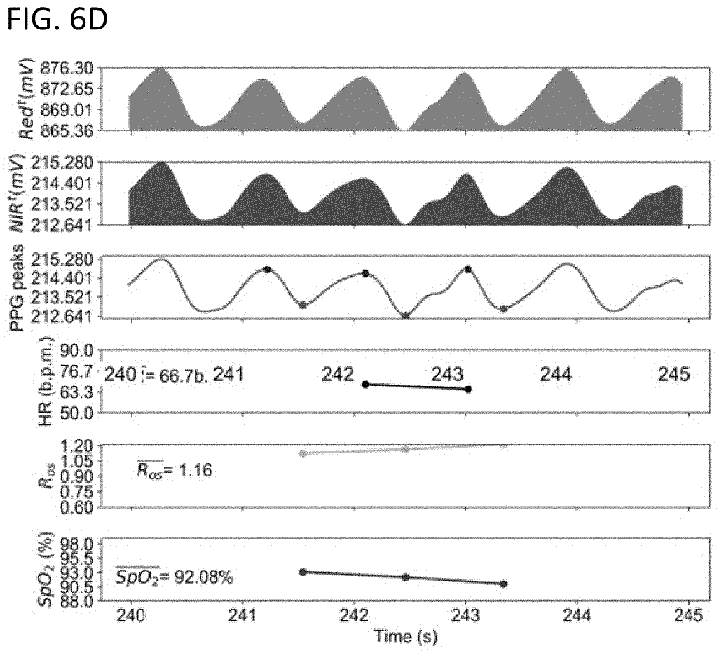

In order to test if the present embodiments can readily detect changes in the oxygen saturation of the body, an altitude simulator is used, which changes the oxygen concentration of the air that the volunteer breathes in through a facemask. The volunteer's oxygen concentration will change accordingly which is picked up by a commercially available finger pulse oximeter probe and the present sensor embodiments under a solar simulator. The readings collected by the prior art oximeter probe are presented in FIGS. 6A,C and the reading collected by a present sensor embodiment in FIGS. 6B,D.

Two spectrally selective OPDs without any programmed light source were used to perform pulse oximetry under ambient light conditions. The ss-OPDs were fabricated by combining OPDs with appropriate filters, which made it possible to obtain green, red and NIR sensitive sensors. These sensors were first tested individually under various ambient light conditions, such as the Sun, fluorescent, LED, or incandescent light, to obtain PPG signals. As a result, it was shown that with proper sensor combinations, it is possible to perform pulse oximetry under all of the ambient light sources that were tested. We took our system outdoors and used two possible combinations, Green+Red and Red+NIR sensors to perform pulse oximetry under the actual Sun. Then our system was used to track changes in the oxygen concentration which was varied by an altitude simulator, value of which was crossed checked by a commercially available pulse oximeter finger probe. The sensors used in our system are compatible with inexpensive large-area production and flexible which will allow healthcare products to be more conformable and affordable. The pulse oximeter with no controlled LEDs is a new concept which can simplify the design of future pulse oximeters, reduce the power consumed by driving the LEDs, make the overall system to be lighter and most of all significantly lower the cost of pulse oximeters.

U.S. Patent Application Publication No. 2017/0156651 A1, which is incorporated herein by reference, discloses various aspects of PPG measurements, including reflectance-based measurements, as well as useful PPG device materials. All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Various embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this specification includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. (canceled)

2. (canceled)

3. (canceled)

4. A pulse oximeter device for conducting PPG measurements using broadband light, comprising:
a first spectrally-selective organic photodiode (ss-OPD) comprising a first spectral filter overlaying a sensing region of a first organic photodiode (OPD), wherein the first OPD absorbs/detects light in a first wavelength range including visible wavelengths up to but not including NIR wavelengths, and the first spectral filter only transmits light having wavelengths including and greater than red wavelengths; and
a second ss-OPD comprising a second spectral filter overlaying a sensing region of a second OPD, wherein the second OPD absorbs/detects light in a second wavelength range including green wavelengths or NIR wavelengths, and the second spectral filter only transmits light having green wavelengths or light having a wavelength of greater than red wavelengths.

5. The pulse oximeter device of claim 4, further comprising a flexible substrate, wherein the first ss-OPD and the second ss-OPD are disposed on the flexible substrate.

6. The pulse oximeter device of claim 5, wherein the first OPD comprises PCDTBT:PCBM70 and the second OPD comprises PCDTBT:PCBM70 or P3HT:O-IDTBR.

7. The pulse oximeter device of claim 4, wherein the first OPD absorbs/detects light in the first wavelength range including visible wavelengths up to about 700 nm, and wherein the first spectral filter only transmits light having a wavelength greater than about 590 nm.

8. The pulse oximeter device of claim 7, wherein the second OPD absorbs/detects light in the second wavelength range including visible wavelengths up to about 700 nm, and wherein the second spectral filter only transmits light in a wavelength range of from about 490 nm to about 570 nm.

9. The pulse oximeter device of claim 7, wherein the second OPD absorbs/detects light in the second wavelength range including wavelengths above about 700 nm up to about 800 nm, and wherein the second spectral filter only transmits light having a wavelength of greater than about 700 nm.

10. (canceled)

11. (canceled)

12. A pulse oximeter device for conducting PPG measurements using broadband light, comprising:
a first organic photodiode (OPD) having a first sensing region, the first OPD absorbs/detects light in a first wavelength range including visible wavelengths up to about 700 nm;
a first optical filter disposed proximal to the first sensing region, the first optical filter transmitting light having a wavelength of greater than about 590 nm a second OPD having a second sensing region, the second OPD absorbs/detects light in a second wavelength range including visible wavelengths and NIR wavelengths greater than 700 nm; and
a second optical filter disposed proximal to the second sensing region, the second optical filter transmitting only light in a wavelength range of from about 490 nm to about 570 nm, or only light having a wavelength of greater than about 700 nm.

13. The device of claim 12, wherein the first OPD comprises PCDTBT:PCBM70 and the second OPD comprises PCDTBT:PCBM70 or P3HT:O-IDTBR.

14. A pulse oximeter device for conducting PPG measurements using broadband light, comprising:
a first organic photodiode (OPD) having a first sensing region, the first OPD absorbs/detects light in a first wavelength range including visible wavelengths up to about 700 nm;
a first optical filter disposed proximal to the first sensing region, the first optical filter transmitting only light having a wavelength of greater than about 590 nm;
a second OPD having a second sensing region, the second OPD absorbs/detects light in a second wavelength range including visible wavelengths; and
a second optical filter disposed proximal to the second sensing region, the second optical filter transmitting light only in a wavelength range of from about 490 nm to about 570 nm.

15. The device of claim 14, wherein the first OPD comprises PCDTBT:PCBM70 and the second OPD comprises PCDTBT:PCBM70.

16. A pulse oximeter device for conducting PPG measurements using broadband light, comprising:
a first organic photodiode (OPD) having a first sensing region, the first OPD absorbs/detects light in a first wavelength range including visible wavelengths up to about 700 nm;
a first optical filter disposed proximal to the first sensing region, the first optical filter transmitting only light having a wavelength of greater than about 590 nm;
a second OPD having a second sensing region the second OPD absorbs/detects light in a second wavelength range including NIR wavelengths; and
a second optical filter disposed proximal to the second sensing region, the second optical filter transmitting only light having a wavelength of greater than about 700 nm.

17. The device of claim 16, wherein the first OPD comprises PCDTBT:PCBM70 and the second OPD comprises P3HT:O-IDTBR.

18. A method of performing PPG measurements using the pulse oximeter device of claim 12, comprising:
positioning the device on a region of interest of a human user;
exposing the device to broadband light; and
obtaining PPG measurements with the device as the pulse oximeter device is exposed to broadband light.

19. The method of claim 18, wherein exposing includes irradiating with a broadband light source selected from the group consisting of a fluorescent lamp, an incandescent lamp, and one or more LEDs.

20. The method of claim 18, wherein the exposing includes exposing the device to sunlight.

21. The method of claim 18, wherein the region of interest includes a finger or a wrist.
